(19) United States
(12) Patent Application Publication
(10) Pub. No.: US 2026/0265069 A1
(43) Pub. Date: Sep. 10, 2026

(54) SPHERICAL CALCIUM SILICATE

(71) Applicant: Evonik Operations GmbH, Essen (DE)

(72) Inventors: Karl W. Gallis, Perryville, MD (US); William J. Hagar, Perryville, MD (US); Fitzgerald A. Sinclair, Smyrna, DE (US); Eric G. Lundquist, Essex, MD (US); Bin Cao, Berwyn, PA (US); Peter Carter, Pittsburgh, PA (US)

(73) Assignee: Evonik Operations GmbH, Essen (DE)

(21) Appl. No.: 18/867,864

(22) PCT Filed: May 24, 2023
(86) PCT No.: PCT/EP2023/063866

§ 371 Date: Nov. 21, 2024

Related U.S. Application Data
(60) Provisional application No. 63/346,627, filed on May 27, 2022.

(30) Foreign Application Priority Data
Jun. 9, 2022 (EP) 22177989.5

(51) Int. Cl. C01B 33/32; A61K 8/02; A61K 8/25

(52) U.S. Cl. CPC C01B 33/325; A61K 8/0216; A61K 8/25; C01P 2004/03; C01P 2004/32; C01P 2004/61; C01P 2006/11; C01P 2006/12; C01P 2006/19; C01P 2006/90

(57) ABSTRACT

A spherical calcium silicate has d50 median particle size from 1 to 35 μm, oil absorption from 40 to 130 mL/100 g, sphericity factor (S₈₀) at least about 0.80, BET surface area between 10-125 m²/g, and % CaO from 0.5-20 wt-%. The spherical calcium silicate are produced by (a) continuously feeding mineral acid and alkali metal silicate into a loop reaction zone having a liquid medium stream, with at least some mineral acid and alkali metal silicate reacting to form silica product; (b) continuously recirculating the liquid medium; (c) continuously discharging a portion of liquid medium with silica product; (d) filtrating and washing the liquid medium with silica product; (e) combining the filtercake of (d) with calcium hydroxide; (f) stirring combined filter cake and calcium hydroxide for 10 min to 180 min and (g) drying. The product can be used in dentifrice compositions, food mixtures, personal care applications and liquid coatings.

### TECHNICAL FIELD

The present disclosure relates generally to new spherical calcium silicates and methods of making and using the same.

### BACKGROUND OF THE INVENTION

Synthetic amorphous calcium silicates have been used in food, feed, pharmaceutical and industrial application areas for many years. They are produced by reacting a previously prepared silica or silicate substantially free of sodium sulfate with a slurry of calcium hydroxide at temperatures of greater than 60° C. (U.S. Pat. Nos. 1,574,363, 4,557,916). The reaction results in the formation of calcium silicate particles that have a high absorptive capacity which is useful in the application areas mentioned above.

U.S. Pat. No. 2,204,113 describes a process of producing finely divided calcium silicate, wherein simultaneously instilling separate aqueous solutions of calcium halide and of a soluble silicate into different locations in a reaction vessel.

Coating compositions, comprising spheroid shaped silica or silicate, are known from US 2012/0216719 A1.

Calcium silicates are also commonly used in personal care applications (U.S. Pat. No. 7,163,669).

Calcium silicates are also known specifically for dental compositions as disclosed in WO 2012/078136 A1 and/or WO 2018/073062 but without defining the main surface characteristics together, such as; BET surface area, oil absorption properties or wt % CaO amount present in the particle.

Silica particles with reduced Relative Dentin Abrasion (RDA) are disclosed and described in US 2020/0206107 A1. Such silica particles can have (i) a d50 median particle size of greater than or equal to about 6 μm, (ii) a ratio of (d90−d10)/d50 in a range from about 1.1 to about 2.4, (iii) a RDA at 20 wt. % loading in a range from about 40 to about 200, and (iv) a sphericity factor (S₈₀) of greater than or equal to about 0.9. These silica particles have a spherical shape or morphology, and can be produced using a continuous loop reactor process.

Therefore, it would be beneficial in oral care to provide calcium silicate materials which increase the soluble calcium levels in saliva and promoting the formation of new hydroxyapatite on the dentin or enamel surface. It can also possibly help small silica particles designed for tubule occlusion to have a higher affinity to the dentin surface and be more likely to remain in the mouth after brushing. Since these silicas are lower in surface area than traditional calcium silicate products, they can also provide mechanical cleaning to the tooth surface as well.

In coating applications the inventive calcium silicate materials can be used to improve the corrosion resistance of metal substrates.

### SUMMARY OF THE INVENTION

This summary is provided to introduce a selection of concepts in a simplified form that are further described below in the detailed description. This summary is not intended to identify required or essential features of the claimed subject matter. Nor is this summary intended to be used to limit the scope of the claimed subject matter.

The inventive spherical calcium silicates that can be used are disclosed and described herein. In accordance with an aspect of this invention, such spherical calcium silicates have (i) a d50 median particle size in a range from 1 to 35 μm, (ii) an oil absorption in a range from 40 to 130 mL/100 g, (iii) a sphericity factor (S₈₀) of greater than or equal to about 0.80, (iv) a BET surface area of between 10-125 m²/g and (v) a % CaO from 0.5-20 wt-%.

These silica and/or silicate particles have a spherical shape or morphology, and can be produced using a continuous loop reactor process.

Also disclosed herein are dentifrice compositions, cosmetics and coatings containing the inventive spherical calcium silicates and methods of using the spherical calcium silicates and compositions.

Both the foregoing summary and the following detailed description provide examples and are explanatory only. Accordingly, the foregoing summary and the following detailed description should not be considered to be restrictive. Further, features or variations may be provided in addition to those set forth herein. For example, certain aspects may be directed to various feature combinations and sub-combinations described in the detailed description.

### BRIEF DESCRIPTION OF THE FIGURES

FIG. 1 Continuous Loop Reactor

FIG. 2 Scanning Electron Micrograph of the silicates of Examples 1A/1B/1C.

FIG. 3 Scanning Electron Micrograph of the silicates of Examples 2A/2B/2C.

FIG. 4 Scanning Electron Micrograph of the silicate of Example 3A.

FIG. 5 Scanning Electron Micrograph of the silica/silicates of Examples 4A/4B/4C/4D.

FIG. 6 Scanning Electron Micrograph of the silica/silicates of Examples 5A/5B/5C.

### DEFINITIONS

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), can be applied, as long as that definition does not conflict with any other disclosure or definition applied herein, or render indefinite or non-enabled any claim to which that definition is applied. To the extent that any definition or usage provided by any document incorporated herein by reference conflicts with the definition or usage provided herein, the definition or usage provided herein controls.

Herein, features of the subject matter are described such that, within particular aspects, a combination of different features can be envisioned. For each and every aspect and each and every feature disclosed herein, all combinations that do not detrimentally affect the designs, compositions, processes, or methods described herein are contemplated and can be interchanged, with or without explicit description of the particular combination. Accordingly, unless explicitly recited otherwise, any aspect or feature disclosed herein can be combined to describe inventive designs, compositions, processes, or methods consistent with the present disclosure.

All publications and patents mentioned herein are incorporated herein by reference for the purpose of describing and disclosing, for example, the constructs and methodologies that are described in the publications, which might be used in connection with the presently described invention.

### DETAILED DESCRIPTION OF THE INVENTION

Disclosed herein are inventive spherical calcium silicate that can be characterized by (i) a d50 median particle size in a range from 1 to 35 μm, preferably 1-20 μm, most preferably 3-15 μm, (ii) an oil absorption in a range from 40 to 130 mL100 g, preferably 50-100 mL100 g, (iii) a sphericity factor (S₈₀) of greater than or equal to about 0.80, preferably greater than 0.85, (iv) a BET surface area of between 10-125 m²/g, preferably 30-80 m²/g, and (v) a % CaO from 0.5-20 wt-%, preferably 3-15 wt-%.

Methods of making these spherical silica and/or silicate particles, dentifrice compositions containing the spherical particles, and methods of treatment using the spherical particles in dentifrice compositions, cosmetics and coatings also are disclosed and described herein.

### Spherical Silicate Particles

Consistent with aspects of the present invention, spherical silicate particles with improved tubule occlusion can have the following characteristics: (i) a d50 median particle size in a range from 1 to 35 μm, (ii) an oil absorption in a range from 40 to 130 mL/100 g, (iii) a sphericity factor (S₈₀) of greater than or equal to 0.80 and (iv) a BET surface area of between 10-125 m²/g and (v) a % CaO from 0.5-20 wt-%. The silicate particles can have any of the characteristics or properties provided below, and in any combination.

The inventive spherical calcium silicate can have a CTAB surface area between 5 and 80 m²/g, preferably between 10 and 70 m²/g.

The inventive spherical calcium silicate can have a pack density between 0.32 and 0.96 g/ml, preferably between 0.40 and 0.80 g/ml.

The inventive spherical calcium silicate can have a 5% pH between 8.0 and 12.0, preferably between 8.5 and 11.0, more preferably between 9.0 and 10.5.

The inventive spherical calcium silicate can have a Ca:Si weight ratio of 0.015 to 0.60, more preferably 0.04 to 0.30, wherein the Ca amount reflects the same amount of CaO % from 0.5-20 wt-%, preferably 3-15 wt-% as given in above and in claim 1.

The inventive spherical calcium silicate can have relatively low water absorption. For instance, the water absorption can be in a range from about 55 to about 115 mL100 g, from about 65 to about 100 mL/100 g, or from about 70 to about 90 mL/100 g. Other appropriate ranges for the water absorption are readily apparent from this disclosure.

The inventive spherical calcium silicate can be amorphous, can be synthetic, or can be both amorphous and synthetic. Moreover, the inventive spherical calcium silicate can comprise precipitated spherical calcium silicate.

The inventive spherical calcium silicate can have a petaloid structure (on the surface by electron microscopy).

### Processes for Producing Spherical Calcium Silicate Particles

The inventive spherical calcium silicate can be produced by the following inventive process.

In general, the inventive process involves (a) continuously feeding a mineral acid and an alkali metal silicate into a loop reaction zone comprising a stream of liquid medium, wherein at least a portion of the mineral acid and the alkali metal silicate react to form a silica product (e.g., the silica and/or silicate particles) in the liquid medium of the loop reaction zone; (b) continuously recirculating the liquid medium through the loop reaction zone; (c) continuously discharging from the loop reaction zone a portion of the liquid medium comprising the silica product; (d) filtrating the liquid medium comprising the silica product and washing the filtercake; (e) combining the filtercake of step (d) with calcium hydroxide, (f) stirring the combined filter cake and calcium hydroxide of step (e) for 10 min to 180 min, preferably 60 min to 120 min (ageing step) and (g) drying the solution.

The inventive spherical calcium silicate disclosed herein are not limited to any particular synthesis procedure. However, in order to achieve the desired sphericity, a continuous loop reactor process for step (a) to (c) can be utilized to form the inventive spherical calcium silicate. This process and associated reactor system (which can include a continuous loop of one or more loop reactor pipes) are described in U.S. Pat. Nos. 8,945,517 and 8,609,068, incorporated herein by reference in their entirety.

Typically, the feed locations of the mineral acid and the alkali metal silicate into the loop reaction zone are different, and the total feed rate of acid and silicate is proportional to, and often equal to, the discharge rate of the liquid medium containing the silica product. All or substantially of the contents within the loop reaction zone are recirculated, for instance, at a rate ranging from about 50 vol. % per minute (the recirculation rate, per minute, is one-half of the total volume of the contents) to about 1000 vol. % per minute (the recirculation rate, per minute, is ten times the total volume of the contents), or from about 75 vol. % per minute to about 500 vol. % per minute.

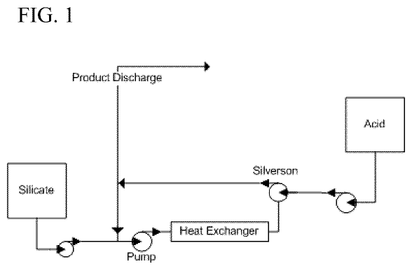

The precipitation apparatus can be configured in a recycle loop where the reaction slurry could be circulated a numerous times before it is discharged (FIG. 1). The loop can comprise of sections of fixed pipe joined together by sections of flexible hose. On one side of the loop a pump can be placed to circulate the reaction mixture and on the opposite side a Silverson in-line mixer can be installed to provide additional shear to the system and also as a convenient place to add the acid. In between the pumps, a static mixer heat exchanger can be installed to provide a means to control the temperature during production of silica. The discharge pipe, located after the acid addition point, allowed the product to discharge as a function of the rates at which silicate and acid are added. The discharge pipe could also be fitted with a back pressure valve that enable the system operate at temperatures greater than 100° C. The product discharge pipe can be oriented to collect product into a tank for additional modification (ex. pH adjustment), or it can be discharged directly into a rotary or press type filter. Optionally, acid can also be added into product discharge line to avoid post synthetic pH adjustments when product is being prepared at pH's greater than 7.0.

In the case of this invention, the Silverson in-line mixer could be modified to provide a high level of mixing without providing shear. This accomplished by removing the stator screen from the Silverson mixer and operating the unit with only the backing plate and the normal mixer head. Alternatively, the Silverson mixer could be run with the standard rotor/square hole high shear stator to obtain a smaller particle size. Particle size could be adjusted in either configuration by changing the Silverson output.

In process step (d) the filtration can be done in a filter press, rotary vacuum filter, belt filter, or similar solid/liquid separation equipment. The washing of the filtercake can be done to reduce the salt residue, e.g. sodium sulfate.

The combining of the filtercake of step (d) with calcium hydroxide in step (e) could be done in an agitated vessel capable of maintaining the desired temperature between 3° and 100° C. The calcium hydroxide could be a slurry with 5 to 20% solid content of calcium hydroxide, more preferably 10-18%. The calcium hydroxide could be a slurry in water.

The stirring in process step (e) could be done at a temperature of 30° C.-100° C. (ageing step) more preferably 60-95° C.

The drying of the calcium silicate (g) could be done in a spray dryer or flash dryer at temperatures sufficient to evaporate the water from the solids.

The inventive spherical calcium silicate can be used in dentifrice compositions, food mixtures, personal care applications and liquid coatings.

### Dentifrice Compositions

The inventive spherical calcium silicate can be used in any suitable composition and for any suitable end-use application, e.g. oral care, personal care, coatings and food areas. Often, the silica and/or silicate particles can be used in oral care applications, such as in a dentifrice composition.

In oral care, calcium can be used for remineralization, by increasing the soluble calcium levels in saliva and promoting the formation of new hydroxyapatite on the dentin or enamel surface. It can also possibly help small silica particles designed for tubule occlusion to have a higher affinity to the dentin surface and be more likely to remain in the mouth after brushing. Since these silicas are lower in surface area than traditional calcium silicate products, they can also provide mechanical cleaning to the tooth surface as well.

The dentifrice composition can contain any suitable amount of the inventive spherical calcium silicate, such as from about 0.5 to about 40 wt. %, from about 1 to about 35 wt. %, from about 3 to about 15 wt. %, from about 3 to about 10 wt. %, of the inventive spherical calcium silicate. These weight percentages are based on the total weight of the dentifrice composition.

The dentifrice composition can be in any suitable form, such as a liquid, powder, or paste.

In addition to the silica and/or silicate particles, the dentifrice composition can contain other ingredients or additives, non-limiting examples of which can include a humectant, a solvent, a binder, a therapeutic agent, a chelating agent, a thickener other than the inventive spherical calcium silicate, a surfactant, an abrasive other than the inventive spherical calcium silicate, a sweetening agent, a colorant, a flavoring agent, a preservative, and the like, as well as any combination thereof.

Humectants serve to add body or “mouth texture” to a dentifrice as well as preventing the dentifrice from drying out. Suitable humectants include polyethylene glycol (at a variety of different molecular weights), propylene glycol, glycerin (glycerol), erythritol, xylitol, sorbitol, mannitol, lactitol, and hydrogenated starch hydrolyzates, and mixtures thereof. In some formulations, humectants are present in an amount from about 20 to about 50 wt. %, based on the weight of dentifrice composition.

A solvent can be present in the dentifrice composition, at any suitable loading, and usually the solvent comprises water. When used, water is preferably deionized and free of impurities, can be present in the dentifrice at loadings from 5 to about 70 wt. %, or from about 5 to about 35 wt. %, based on the weight of dentifrice composition.

Therapeutic agents also can be used in the compositions of this invention to provide for the prevention and treatment of dental caries, periodontal disease, and temperature sensitivity, for example. Suitable therapeutic agents can include, but are not limited to, fluoride sources, such as sodium fluoride, sodium monofluorophosphate, potassium monofluorophosphate, stannous fluoride, potassium fluoride, sodium fluorosilicate, ammonium fluorosilicate and the like; condensed phosphates such as tetrasodium pyrophosphate, tetrapotassium pyrophosphate, disodium dihydrogen pyrophosphate, trisodium monohydrogen pyrophosphate; tripolyphosphates, hexametaphosphates, trimetaphosphates and pyrophosphates; antimicrobial agents such as triclosan, bisguanides, such as alexidine, chlorhexidine and chlorhexidine gluconate; enzymes such as papain, bromelain, glucoamylase, amylase, dextranase, mutanase, lipases, pectinase, tannase, and proteases; quaternary ammonium compounds, such as benzalkonium chloride (BZK), benzethonium chloride (BZT), cetylpyridinium chloride (CPC), and domiphen bromide; metal salts, such as zinc citrate, zinc chloride, and stannous fluoride; sanguinaria extract and sanguinarine; volatile oils, such as eucalyptol, menthol, thymol, and methyl salicylate; amine fluorides; peroxides and the like. Therapeutic agents can be used in dentifrice formulations singly or in combination, and at any therapeutically safe and effective level or dosage.

Thickening agents are useful in the dentifrice compositions to provide a gelatinous structure that stabilizes the toothpaste against phase separation. Suitable thickening agents include silica thickener; starch; glycerite of starch; gums such as gum karaya (sterculia gum), gum tragacanth, gum arabic, gum ghatti, gum acacia, xanthan gum, guar gum and cellulose gum; magnesium aluminum silicate (Veegum); carrageenan; sodium alginate; agar-agar; pectin; gelatin; cellulose compounds such as cellulose, carboxymethyl cellulose, hydroxyethyl cellulose, hydroxypropyl cellulose, hydroxymethyl cellulose, hydroxymethyl carboxypropyl cellulose, methyl cellulose, ethyl cellulose, and sulfated cellulose; (co)polymers of acrylic acid, natural and synthetic clays such as hectorite clays; and mixtures thereof. Typical levels of thickening agents or binders are up to about 15 wt. % of a toothpaste or dentifrice composition.

Useful silica thickeners for utilization within a toothpaste composition, for example, include, as a non-limiting example, an amorphous precipitated silica such as ZEODENT® 165 silica. Other non-limiting silica thickeners include ZEODENT® 153, 163 and/or 167 and ZEOFREE® 177 and/or 265 silica products, all available from Evonik Corporation.

Surfactants can be used in the dentifrice compositions of the invention to make the compositions more cosmetically acceptable. The surfactant is preferably a detersive material which imparts to the composition detersive and foaming properties. Suitable surfactants are safe and effective amounts of anionic, cationic, nonionic, zwitterionic, amphoteric and betaine surfactants such as sodium lauryl sulfate, sodium dodecyl benzene sulfonate, alkali metal or ammonium salts of lauroyl sarcosinate, myristoyl sarcosinate, palmitoyl sarcosinate, stearoyl sarcosinate and oleoyl sarcosinate, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocoamidopropyl betaine, lauramidopropyl betaine, palmityl betaine and the like. Sodium lauryl sulfate is a preferred surfactant. The surfactant is typically present in the compositions of the present invention in an amount from about 0.1 to about 15 wt. %, from about 0.3 to about 5 wt. %, or from about 0.3 to about 2.5 wt. %.

The disclosed spherical calcium silicate can be utilized alone as the abrasive in the dentifrice composition, or as an additive or co-abrasive with other abrasive materials discussed herein or known in the art. Thus, any number of other conventional types of abrasive additives can be present within the dentifrice compositions of the invention. Other such abrasive particles include, for example, precipitated calcium carbonate (PCC), ground calcium carbonate (GCC), chalk, bentonite, dicalcium phosphate or its dihydrate forms, silica gel (by itself, and of any structure), precipitated silica, amorphous precipitated silica (by itself, and of any structure as well), perlite, titanium dioxide, dicalcium phosphate, calcium pyrophosphate, alumina, hydrated alumina, calcined alumina, aluminum silicate, insoluble sodium metaphosphate, insoluble potassium metaphosphate, insoluble magnesium carbonate, zirconium silicate, particulate thermosetting resins and other suitable abrasive materials. Such materials can be introduced into the dentifrice compositions to tailor the polishing characteristics of the target formulation.

Sweeteners can be added to the dentifrice composition (e.g., toothpaste) to impart a pleasing taste to the product. Suitable sweeteners include saccharin (as sodium, potassium or calcium saccharin), cyclamate (as a sodium, potassium or calcium salt), acesulfame-K, thaumatin, neohesperidin dihydrochalcone, ammoniated glycyrrhizin, dextrose, levulose, sucrose, mannose, and glucose.

Colorants can be added to improve the aesthetic appearance of the product. Suitable colorants include without limitation those colorants approved by appropriate regulatory bodies such as the FDA and those listed in the European Food and Pharmaceutical Directives and include pigments, such as TiO₂, and colors such as FD&C and D&C dyes.

Flavoring agents also can be added to dentifrice compositions. Suitable flavoring agents include, but are not limited to, oil of wintergreen, oil of peppermint, oil of spearmint, oil of sassafras, and oil of clove, cinnamon, anethole, menthol, thymol, eugenol, eucalyptol, lemon, orange and other such flavor compounds to add fruit notes, spice notes, etc. These flavoring agents generally comprise mixtures of aldehydes, ketones, esters, phenols, acids, and aliphatic, aromatic and other alcohols.

Preservatives also can be added to the compositions of the present invention to prevent bacterial growth. Suitable preservatives approved for use in oral compositions such as methylparaben, propylparaben and sodium benzoate can be added in safe and effective amounts.

Other ingredients can be used in the dentifrice composition, such as desensitizing agents, healing agents, other caries preventative agents, chelating/sequestering agents, vitamins, amino acids, proteins, other anti-plaque/anti-calculus agents, opacifiers, antibiotics, anti-enzymes, enzymes, pH control agents, oxidizing agents, antioxidants, and the like.

### Methods of Use

Any of the inventive spherical calcium silicate and any of the compositions disclosed herein can be used in methods of treatment. For instance, a method of reducing dental sensitivity consistent with this invention can comprise contacting any of the spherical calcium silicate (or any of the compositions) disclosed herein with a surface of a mammalian tooth. Thus, the spherical calcium silicate (or compositions) can be applied to, or delivered to, the surface of the mammalian tooth via brushing or any other suitable technique. Any suitable amount of the silica and/or silicate particles (or compositions) can be used, and for any appropriate period of time.

In another aspect, a method for occluding a dentin tubule within a surface of a mammalian tooth consistent with this invention can comprise contacting any of the spherical calcium silicate (or any of the compositions) disclosed herein with the surface of the mammalian tooth. As above, any suitable amount of the spherical calcium silicate (or compositions) can be applied to, or delivered to, the surface of the mammalian tooth via brushing or any other suitable technique, and for any appropriate period of time.

The sphericity factor (S₈₀) is determined as follows. Scanning electron microscopy images were taken on a Zeiss Sigma instrument equipped with a field emission detector. Samples were dispersed in methanol and then the methanol slurry was dried on an aluminum sample holder. The dry samples were sputter coated with platinum to minimize charging before images were taken.

An SEM image of the silica and/or silicate particle sample is magnified 250-2,000 times, which is representative of the silica and/or silicate particle sample, and is imported into photo imaging software, and the outline of each particle (two-dimensionally) is traced. Particles that are close in proximity to one another but not attached to one another should be considered separate particles for this analysis. The outlined particles are then filled in with color, and the image is imported into particle characterization software (e.g., IMAGE-PRO PLUS available from Media Cybernetics, Inc., Bethesda, Md.) capable of determining the perimeter and area of the particles. Sphericity of the particles can then be calculated according to the equation, Sphericity=(perimeter)² divided by (4π×area), wherein perimeter is the software measured perimeter derived from the outlined trace of the particles, and wherein area is the software measured area within the traced perimeter of the particles.

The sphericity calculation is performed for each particle that fits entirely within the SEM image. These values are then sorted by value, and the lowest 20% of these values are discarded. The remaining 80% of these values are averaged to obtain the sphericity factor (S₈₀). Additional information on sphericity can be found in U.S. Pat. Nos. 8,945,517 and 8,609,068, incorporated herein by reference in their entirety.

The BET surface areas disclosed herein were determined on a Micromeritics TriStar II 3020 V1.03 using the BET nitrogen adsorption method of Brunaur et al., J. Am. Chem. Soc., 60, 309 (1938), and such technique is well known to those skilled in the art.

CTAB surface areas disclosed herein were determined by absorption of CTAB (cetyltrimethylammonium bromide) on the silica surface, the excess separated by centrifugation and the quantity determined by titration with sodium lauryl sulfate using a surfactant electrode. Specifically, about 0.5 grams of the silica and/or silicate particles were placed in a 250-mL beaker with 100 mL CTAB solution (5.5 g/L), mixed on an electric stir plate for 1 hour, then centrifuged for 30 min at 10,000 RPM. One mL of 10% Triton X-100 was added to 5 mL of the clear supernatant in a 100-mL beaker. The pH was adjusted to 3-3.5 with 0.1 N HCl and the specimen was titrated with 0.01 M sodium lauryl sulfate using a surfactant electrode (Brinkmann SUR1501-DL) to determine the endpoint.

The d50 median particle size refers to the particle size for which 50% of the sample has a smaller size and 50% of the sample has a larger size. Median particle size (d50), mean particle size (average), and d95 were determined via the laser diffraction method using a Horiba LA 300 instrument. Dry particles were submitted to the instrument for analysis.

For pour density and pack density, 20 grams of the sample were placed into a 250 mL graduated cylinder with a flat rubber bottom. The initial volume was recorded and used to calculate the pour density by dividing it into the weight of sample used. The cylinder was then placed onto a tap density machine where it was rotated on a cam at 60 RPM. The cam is designed to raise and drop the cylinder a distance of 5.715 cm once per second, until the sample volume is constant, typically for 15 min. This final volume is recorded and used to calculate the packed density by dividing it into the weight of sample used.

Oil absorption values were determined in accordance with the rub-out method described in ASTM D281 using linseed oil (cc oil absorbed per 100 g of the particles). Generally, a higher oil absorption level indicates a particle with a higher level of large pore porosity, also described as higher structure.

Water absorption values were determined with an Absorptometer “C” torque rheometer from C.W. Brabender Instruments, Inc. Approximately ⅓ of a cup of the silica sample was transferred to the mixing chamber of the Absorptometer and mixed at 150 RPM. Water then was added at a rate of 6 mL/min, and the torque required to mix the powder was recorded. As water is absorbed by the powder, the torque will reach a maximum as the powder transforms from free-flowing to a paste. The total volume of water added when the maximum torque was reached was then standardized to the quantity of water that can be absorbed by 100 g of powder. Since the powder was used on an as received basis (not previously dried), the free moisture value of the powder was used to calculate a “moisture corrected water AbC value” by the following equation.

$$Water Absorption = \frac{water absorbed ( cc ) + \% moisture}{( 100 ( g ) - \% moisture ) / 100}$$

The Absorptometer is commonly used to determine the oil number of carbon black in compliance with ASTM D 2414 methods B and C and ASTM D 3493.

The pH values disclosed herein (5% pH) were determined in an aqueous system containing 5 wt. % solids in deionized water using a pH meter.

The % CaO (calcium concentrations) were determined by the following method. 2.0000 g of silica was wet with a few drops of deionized water in a platinum crucible. 10 ml of perchloric acid (72%) and 10 ml of hydrofluoric acid (48-50%) were added and the platinum dish was slowly heated on a stir plate in a fume hood. As the platinum dish was heated, dense white fumes were evolved. The sides of the crucible were then carefully rinsed with boric acid (4%) and it was also heated to fumes. After cooling, the contents of the crucible were transferred to a 250 ml volumetric flask and the crucible was washed with deionized water to make sure all remaining contents were quantitatively transferred. The dish was then rinsed with 5 ml of hydrochloric acid (36%) and the washings were also added to the volumetric flask. Approximately 200 ml of deionized water were added to the volumetric flask, and if the resulting solution was cloudy, it was heated on a low temperature hot plate until it became clear. After cooling, 2.50 ml of a scandium internal standard solution was added and the volumetric flask was filled to the mark with deionized water. The concentrations of the metals in the solution were then determined by ICP/OES.

Sulfate concentrations were measured on a LECO SC832 series combustion analyzer from LECO, St. Joseph, Michigan, USA. The samples were placed in a combustion boat and heated to approximately 1350° C. under an oxygen rich environment. Carbon and sulfur are released as their respective oxides and are measured by IR spectroscopy. The concentration of sulfur or carbon is determined by comparison with known standards.

### EXAMPLES

The invention is further illustrated by the following examples, which are not to be construed in any way as imposing limitations to the scope of this invention. Various other aspects, modifications, and equivalents thereof which, after reading the description herein, may suggest themselves to one of ordinary skill in the art without departing from the spirit of the present invention or the scope of the appended claims.

### Continuous Loop Reactor Set-Up:

The precipitation apparatus was configured in a recycle loop where the reaction slurry could be circulated a numerous times before it is discharged (FIG. 1). The loop was comprised of sections of fixed pipe joined together by sections of flexible hose. The internal diameter of the piping/hose was approximately 1.5″ with a volume of approximately 45 L. On one side of the loop a pump was placed to circulate the reaction mixture and on the opposite side a Silverson in-line mixer was installed to provide additional shear to the system and also as a convenient place to add the acid. In between the pumps, a static mixer heat exchanger was installed to provide a means to control the temperature during production of silica. The discharge pipe, located after the acid addition point, allowed the product to discharge as a function of the rates at which silicate and acid are added. The discharge pipe could also be fitted with a back pressure valve that enable the system operate at temperatures greater than 100° C. The product discharge pipe can be oriented to collect product into a tank for additional modification (ex. pH adjustment), or it can be discharged directly into a rotary or press type filter. Optionally, acid can also be added into product discharge line to avoid post synthetic pH adjustments when product is being prepared at pH's greater than 7.0.

In the case of this invention, the Silverson in-line mixer could be modified to provide a high level of mixing without providing shear. This was accomplished by removing the stator screen from the Silverson mixer and operating the unit with only the backing plate and the normal mixer head (Example D). Alternatively, the Silverson mixer could be run with the standard rotor/square hole high shear stator to obtain a smaller particle size (Example C). Particle size could be adjusted in either configuration by changing the Silverson output.

### Initial Set-Up

Prior to the introduction of acid and silicate into the system, precipitated silica, sodium sulfate, sodium silicate and water were added and recirculated at 180 L/min. This step was performed to fill the recycle loop with the approximate contents and concentrations of a typical batch to minimize the purging time before the desired product could be collected. It was also done to avoid the possibility of forming gel in the reactor, although subsequent experimentation has revealed that acid and silicate could be directly added to the loop filled with water without gelling or plugging the system.

### Example 1

1.5 kg of Zeodent® 103, 1.34 kg of sodium sulfate, 11.1 L of sodium silicate (3.32 MR, 19.5%) and 20 L of water were added to the recirculation loop and it was heated to 90° C. with recirculation at 180 L/min with the Silverson operating at 2320 Hz (1740 RPM) with the stator removed and a shear frequency of (5,000—reference 145 patent application). Sodium silicate (3.32 MR, 19.5%) and sulfuric acid (17.1%) were added simultaneously to the loop at a silicate rate of 1.7 L/min and an acid rate sufficient to maintain a pH of 7.5. If necessary, the acid rate was adjusted accordingly to maintain the pH. Acid and silicate were added under these conditions for 40 minutes to purge unwanted silica out of the system before the desired material was collected. After 40 minutes had passed, the collection vessel was emptied and its contents discarded. The silica product was then collected in a vessel with stirring at 40 RPM while maintaining the temperature at approximately 80° C. After the desired quantity of product was collected, addition of acid and silicate were stopped and the contents of the loop were filtered to a conductivity less than 1000 μS/cm.

### Example 1A

30 kg of silica wet cake (based on dry mass) from Example 1 and 120 L of water were combined and stirred in a stainless-steel tank. To this, 8.7 L of a slurry of calcium hydroxide with a specific gravity of 1.104 g/mL and an approximate solids of 15.6% was added to the diluted silica slurry and the mixture was stirred for 60 minutes. After 60 minutes, the entire solution was spray dried to a target moisture of ~5%.

### Example 1B

30 kg of silica wet cake (based on dry mass) from Example 1 and 120 L of water were combined and stirred in a stainless-steel tank. To this, 17.4 L of a slurry of calcium hydroxide with a specific gravity of 1.104 g/mL and an approximate solids of 15.6% was added to the diluted silica slurry and the mixture was stirred for 60 minutes. After 60 minutes, the entire solution was spray dried to a target moisture of ~5%.

### Example 1C

30 kg of silica wet cake (based on dry mass) from Example 1 and 120 L of water were combined and stirred in a stainless-steel tank. To this, 26.1 L of a slurry of calcium hydroxide with a specific gravity of 1.104 g/mL and an approximate solids of 15.6% was added to the diluted silica slurry and the mixture was stirred for 60 minutes. After 60 minutes, the entire solution was spray dried to a target moisture of ~5%.

### Example 2

1.5 kg of Zeodent® 103, 1.34 kg of sodium sulfate, 11.1 L of sodium silicate (3.32 MR, 19.5%) and 20 L of water were added to the recirculation loop and it was heated to 70° C. with recirculation at 180 L/min with the Silverson operating at 30 Hz (1740 RPM) with the stator removed and a shear frequency of (5,000—reference same as above). Sodium silicate (3.32 MR, 19.5%) and sulfuric acid (17.1%) were added simultaneously to the loop at a silicate rate of 1.7 L/min and an acid rate sufficient to maintain a pH of 7.5. If necessary, the acid rate was adjusted accordingly to maintain the pH. Acid and silicate were added under these conditions for 40 minutes to purge unwanted silica out of the system before the desired material was collected. After 40 minutes had passed, the collection vessel was emptied and its contents discarded. The silica product was then collected in a vessel with stirring at 40 RPM while maintaining the temperature at approximately 80° C. After the desired quantity of product was collected, addition of acid and silicate were stopped and the contents of the loop were allowed to circulate.

### Example 2A

30 kg of silica wet cake (based on dry mass) from Example 2 and 120 L of water were combined and stirred in a stainless-steel tank. To this, 3.7 L of a slurry of calcium hydroxide with a specific gravity of 1.120 g/mL and an approximate solids of 18.2% was added to the diluted silica slurry and the mixture was stirred for 60 minutes. After 60 minutes, the entire solution was spray dried to a target moisture of ~5%.

### Example 2B

30 kg of silica wet cake (based on dry mass) from Example 2 and 120 L of water were combined and stirred in a stainless-steel tank. To this, 7.4 L of a slurry of calcium hydroxide with a specific gravity of 1.120 g/mL and an approximate solids of 18.2% was added to the diluted silica slurry and the mixture was stirred for 60 minutes. After 60 minutes, the entire solution was spray dried to a target moisture of ~5%.

### Example 2C

30 kg of silica wet cake (based on dry mass) from Example 2 and 120 L of water were combined and stirred in a stainless-steel tank. To this, 14.9 L of a slurry of calcium hydroxide with a specific gravity of 1.120 g/mL and an approximate solids of 18.2% was added to the diluted silica slurry and the mixture was stirred for 60 minutes. After 60 minutes, the entire solution was spray dried to a target moisture of ~5%.

### Example 3

1.5 kg of Zeodent® 103, 1.34 kg of sodium sulfate, 11.1 L of sodium silicate (3.32 MR, 19.5%) and 20 L of water were added to the recirculation loop and it was heated to 90° C. with recirculation at 180 L/min with the Silverson operating at 2320 Hz (1740 RPM) with the stator removed and a shear frequency of (5,000—reference 145 patent application). Sodium silicate (3.32 MR, 19.5%) and sulfuric acid (17.1%) were added simultaneously to the loop at a silicate rate of 1.7 L/min and an acid rate sufficient to maintain a pH of 7.5. If necessary, the acid rate was adjusted accordingly to maintain the pH. Acid and silicate were added under these conditions for 40 minutes to purge unwanted silica out of the system before the desired material was collected. After 40 minutes had passed, the collection vessel was emptied and its contents discarded. The silica product was then collected in a vessel with stirring at 40 RPM while maintaining the temperature at approximately 80° C. After the desired quantity of product was collected, addition of acid and silicate were stopped and the contents of the loop were filtered to a conductivity less than 1000 μS/cm.

### Example 3A

The filter cake from Example 3 was diluted to 10% solids and was pumped into a 400 gallon batch reactor and was stirred at 60 RPM. This slurry contained approximately 45 kg of silica by dry mass. 11.1 L of a slurry of calcium hydroxide at a specific gravity of 1.054 g/mL and an approximate solids of 18.2% was added to the silica slurry and the reactor was heated to 95° and was then aged for 2 hours. After the 2 hour ageing time, the batch contents were spray dried to a target moisture of 5%.

The chemical data and physical data are shown in Table 1 and 2.

TABLE 1
Chemical Data

| | | Ca:Si | | | | | |
|---|---|---|---|---|---|---|---|
| | % CaO | (wt | Al₂O₃ | MgO | Na₂O | TiO₂ | LECO SO₄ |
| Example | (wt-%) | ratio) | (%) | (ppm) | (%) | (ppm) | (%) |
| 1A | 3.0 | 0.0505 | 0.13 | 350 | 1.89 | 139 | 0.25 |
| 1B | 1.3 | 0.0209 | 0.11 | 190 | 1.94 | 149 | 0.25 |
| 1C | 1.2 | 0.0193 | 0.11 | 181 | 2.12 | 157 | 0.23 |
| 2A | 1.7 | 0.0279 | 903 | 239 | 1.97 | 174 | 0.22 |
| 2B | 2.9 | 0.0495 | 871 | 353 | 1.69 | 157 | 0.22 |
| 2C | 5.0 | 0.0915 | 961 | 570 | 1.73 | 166 | 0.21 |
| 3A | 2.8 | 0.0474 | 949 | 338 | 4.98 | 183 | 0.26 |

TABLE 2
Physical Data

| | | | | | d50 | | |
|---|---|---|---|---|---|---|---|
| | | | BET | CTAB | Median | | |
| | S₈₀ | Oil | Surface | Surface | Particle | Pack | |
| | Sphericity | Absorption | Area | Area | Size | Density | 5% |
| Example | factor | (cc/100 g) | (m²/g) | (m²/g) | (μm) | (g/mL) | pH |
| 1A | 0.81 | 83 | 54 | 48 | 23.7 | 0.62 | 10.5 |
| 1B | 0.83 | 55 | 46 | 37 | 32.8 | 0.78 | 10.4 |
| 1C | 0.83 | 47 | 35 | 30 | 33.3 | 0.82 | 10.5 |
| 2A | 0.80 | 65 | 120 | 67 | 27.7 | 0.70 | 10.2 |
| 2B | 0.81 | 62 | 73 | 56 | 29.3 | 0.70 | 10.4 |
| 2C | 0.85 | 62 | 73 | 59 | 24.9 | 0.67 | 10.8 |
| 3A | 0.84 | 78 | 15 | 10 | 26.0 | 0.67 | 11.8 |

### Example 4

### Comparative Example 4A

1.5 kg of Zeodent® 103, 1.34 kg of sodium sulfate, 11.1 L of sodium silicate (3.32 MR, 19.5%) and 20 L of water were added to the recirculation loop and it was heated to 83° C. with recirculation at 180 L/min with the stator installed in the Silverson. Sodium silicate (3.32 MR, 13.3%) and sulfuric acid (11.4%) were added simultaneously to the loop at a silicate rate of 6.4 L/min and an acid rate sufficient to maintain a pH of 7.5. If necessary, the acid rate was adjusted accordingly to maintain the pH. Acid and silicate were added under these conditions for 40 minutes to purge unwanted silica out of the system before the desired material was collected. After 40 minutes had passed, the collection vessel was emptied and its contents discarded. The silica product was then collected in a vessel with stirring at 40 RPM while maintaining the temperature at approximately 80° C. After the desired quantity of product was collected, addition of acid and silicate were stopped and the contents of the loop were filtered to a conductivity less than 1000 μS/cm and was then spray dried to a target moisture of 5%.

### Comparative Example 4B

200 g of Comparative Example 4A was slurried in water at a concentration of 10% and was heated to 60° C. with overhead stirring at 300 RPM (sufficient to keep the silica from settling). 64 ml of an aqueous calcium hydroxide slurry (17.5% solids, 1.12 g/ml) was added to the silica slurry and the reaction mixture was aged 45 minutes with stirring at 300 RPM. After the 45 minutes, the reaction mixture was filtered and was oven dried at 105° C. overnight.

### Comparative Example 4C

200 g of Comparative Example 4A was slurried in water at a concentration of 10% and was heated to 60° C. with overhead stirring at 300 RPM (sufficient to keep the silica from settling). 86 ml of an aqueous calcium hydroxide slurry (17.5% solids, 1.12 g/ml) was added to the silica slurry and the reaction mixture was aged 45 minutes with stirring at 300 RPM. After the 45 minutes, the reaction mixture was filtered and was oven dried at 105° C. overnight.

### Comparative Example 4D

200 g of Comparative Example 4A was slurried in water at a concentration of 10% and was heated to 60° C. with overhead stirring at 300 RPM (sufficient to keep the silica from settling). 110 ml of an aqueous calcium hydroxide slurry (17.5% solids, 1.12 g/ml) was added to the silica slurry and the reaction mixture was aged 45 minutes with stirring at 300 RPM. After the 45 minutes, the reaction mixture was filtered and was oven dried at 105° C. overnight.

### Example 5

### Comparative Example 5A

1.5 kg of Zeodent® 103, 1.34 kg of sodium sulfate, 11.1 L of sodium silicate (3.32 MR, 19.5%) and 20 L of water were added to the recirculation loop and it was heated to 90° C. with recirculation at 180 L/min with the Silverson operating at 2320 Hz (1740 RPM) with the stator removed and a shear frequency of (5,000—reference 145 patent application). Sodium silicate (3.32 MR, 19.5%) and sulfuric acid (17.1%) were added simultaneously to the loop at a silicate rate of 1.7 L/min and an acid rate sufficient to maintain a pH of 7.5. If necessary, the acid rate was adjusted accordingly to maintain the pH. Acid and silicate were added under these conditions for 40 minutes to purge unwanted silica out of the system before the desired material was collected. After 40 minutes had passed, the collection vessel was emptied and its contents discarded. The silica product was then collected in a vessel with stirring at 40 RPM while maintaining the temperature at approximately 80° C. After the desired quantity of product was collected, addition of acid and silicate were stopped and the contents of the loop was filtered to a conductivity less than 1000 μS/cm and was then spray dried to a target moisture of 5%.

### Example 5B

200 g of Comparative Example 5A was slurried in water at a concentration of 10% and was heated to 60° C. with overhead stirring at 300 RPM (sufficient to keep the silica from settling). 64 ml of an aqueous calcium hydroxide slurry (17.5% solids, 1.12 g/ml) was added to the silica slurry and the reaction mixture was aged 2.5 hours with stirring at 300 RPM. After the 45 minutes, the reaction mixture was filtered and was oven dried at 105° C. overnight.

### Example 5C

200 g of Comparative Example 5A was slurried in water at a concentration of 10% and was heated to 60° C. with overhead stirring at 300 RPM (sufficient to keep the silica from settling). 110 ml of an aqueous calcium hydroxide slurry (17.5% solids, 1.12 g/ml) was added to the silica slurry and the reaction mixture was aged 2.5 hours with stirring at 300 RPM. After the 45 minutes, the reaction mixture was filtered and was oven dried at 105° C. overnight.

The chemical data and physical data are shown in Table 3.

TABLE 3

| | | | | | | | d50 | | | |
|---|---|---|---|---|---|---|---|---|---|---|
| | | | | | BET | CTAB | Median | | | |
| | S₈₀ | % | Ca:Si | AbC | Surface | Surface | Particle | Pack | Oil | |
| | Sphericity | CaO | (wt | (cc/ | area | area | Size | Density | Absorption | pH |
| Example | factor | (wt-%) | ratio) | 100 g) | (m²/g) | (m²/g) | (μm) | (g/mL) | (mL/−100 g) | (5%) |
| 4A | n/a | 0.02 | 0.0003 | 83 | 100 | 70 | 7.9 | 0.59 | 86 | 7.3 |
| 4B | 0.74 | 3.6 | 0.0605 | 100 | 41 | 41 | 5.6 | 0.50 | 95 | 9.7 |
| 4C | 0.79 | 4.8 | 0.0833 | 126 | 61 | 54 | 7.4 | 0.51 | 123 | 9.7 |
| 4D | | 5.9 | 0.1084 | 137 | 68 | 60 | 8.2 | 0.32 | 130 | 9.6 |
| 5A | n/a | 0.01 | 0.0002 | 52 | 40 | 37 | 9.7 | 0.95 | 51 | 8.0 |
| 5B | 0.86 | 3.8 | 0.0638 | 92 | 49 | 47 | 11.8 | 0.50 | 77 | 9.7 |
| 5C | 0.89 | 6.5 | 0.1133 | 124 | 75 | 58 | 14.3 | 0.43 | 99 | 9.7 |

### Example 6

1.5 kg of Zeodent® 103, 1.34 kg of sodium sulfate, 11.1 L of sodium silicate (3.32 MR, 19.5%) and 20 L of water were added to the recirculation loop and it was heated to 83° C. with recirculation at 180 L/min with the stator installed in the Silverson. Sodium silicate (3.32 MR, 13.3%) and sulfuric acid (11.4%) were added simultaneously to the loop at a silicate rate of 6.4 L/min and an acid rate sufficient to maintain a pH of 7.5. If necessary, the acid rate was adjusted accordingly to maintain the pH. Acid and silicate were added under these conditions for 40 minutes to purge unwanted silica out of the system before the desired material was collected. After 40 minutes had passed, the collection vessel was emptied and its contents discarded. The silica product was then collected in a vessel with stirring at 40 RPM while maintaining the temperature at approximately 80° C. After the desired quantity of product was collected, addition of acid and silicate were stopped and the contents of the loop were filtered to a conductivity less than 1000 μS/cm.

### Example 6A

30 kg of silica wet cake (based on dry mass) from Example 6 and 120 L of water were combined and stirred in a stainless-steel tank. To this, a slurry of calcium hydroxide containing 1.5 kg of calcium hydroxide (by dry mass) was added to the diluted silica slurry and the mixture was stirred for 60 minutes at ambient temperature. After 60 minutes, the entire solution was spray dried to a target moisture of ~5%.

### Example 6B

30 kg of silica wet cake (based on dry mass) from Example 6 and 120 L of water were combined and stirred in a stainless-steel tank. To this, a slurry of calcium hydroxide containing 4.5 kg of calcium hydroxide (by dry mass) was added to the diluted silica slurry and the mixture was stirred for 60 minutes at ambient temperature. After 60 minutes, the entire solution was spray dried to a target moisture of ~5%.

### Example 7

1.5 kg of Zeodent® 103, 1.34 kg of sodium sulfate, 11.1 L of sodium silicate (3.32 MR, 19.5%) and 20 L of water were added to the recirculation loop and it was heated to 70° C. with recirculation at 180 L/min with the stator installed in the Silverson. Sodium silicate (3.32 MR, 13.3%) and sulfuric acid (11.4%) were added simultaneously to the loop at a silicate rate of 6.4 L/min and an acid rate sufficient to maintain a pH of 7.5. If necessary, the acid rate was adjusted accordingly to maintain the pH. Acid and silicate were added under these conditions for 40 minutes to purge unwanted silica out of the system before the desired material was collected. After 40 minutes had passed, the collection vessel was emptied and its contents discarded. The silica product was then collected in a vessel with stirring at 40 RPM while maintaining the temperature at approximately 80° C. After the desired quantity of product was collected, addition of acid and silicate were stopped and the contents of the loop were filtered to a conductivity less than 1000 μS/cm.

### Example 7A

30 kg of silica wet cake (based on dry mass) from Example 6 and 120 L of water were combined and stirred in a stainless-steel tank. To this, a slurry of calcium hydroxide containing 4.5 kg of calcium hydroxide (by dry mass) was added to the diluted silica slurry and the mixture was stirred for 60 minutes at ambient temperature. After 60 minutes, the entire solution was spray dried to a target moisture of ~5%.

### Example 7B

30 kg of silica wet cake (based on dry mass) from Example 6 and 120 L of water were combined and stirred in a stainless-steel tank. To this, a slurry of calcium hydroxide containing 6.0 kg of calcium hydroxide (by dry mass) was added to the diluted silica slurry and the mixture was stirred for 60 minutes at ambient temperature. After 60 minutes, the entire solution was spray dried to a target moisture of ~5%.

### Example 8

1.5 kg of Zeodent® 103, 1.34 kg of sodium sulfate, 11.1 L of sodium silicate (3.32 MR, 19.5%) and 20 L of water were added to the recirculation loop and it was heated to 83° C. with recirculation at 180 L/min with the stator installed in the Silverson. Sodium silicate (3.32 MR, 13.3%) and sulfuric acid (11.4%) were added simultaneously to the loop at a silicate rate of 6.4 L/min and an acid rate sufficient to maintain a pH of 7.5. If necessary, the acid rate was adjusted accordingly to maintain the pH. Acid and silicate were added under these conditions for 40 minutes to purge unwanted silica out of the system before the desired material was collected. After 40 minutes had passed, the collection vessel was emptied and its contents discarded. The silica product was then collected in a vessel with stirring at 40 RPM while maintaining the temperature at approximately 80° C. After the desired quantity of product was collected, addition of acid and silicate were stopped and the contents of the loop were filtered to a conductivity less than 1000 μS/cm.

### Example 8A

The filter cake from Example 8 was diluted to 10% solids and was pumped into a 400 gallon batch reactor and was stirred at 60 RPM. This slurry contained approximately 45 kg of silica by dry mass. 39.1 L of a slurry of calcium hydroxide with a specific gravity of 1.104 g/mL and an approximate solids of 15.6% was added to the silica slurry and the reactor was heated to 95° and was then aged for 2 hours. After the 2 hour ageing time, the batch contents were spray dried to a target moisture of 5%.

The chemical data and physical data are shown in Table 4 and Table 5.

TABLE 4
Chemical data

| | S₈₀ | | Ca/Si | | | | | LECO |
|---|---|---|---|---|---|---|---|---|
| | Sphericity | % CaO | (wt | Al₂O₃ | MgO | Na₂O | TiO₂ | SO₄ |
| Example | factor | (wt-%) | ratio) | (%) | (ppm) | (%) | (ppm) | (%) |
| 6A | 0.88 | 3.0 | 0.0523 | 0.13 | 336 | 0.41 | 149 | 0.29 |
| 6B | 0.88 | 7.4 | 0.1559 | 0.12 | 823 | 0.40 | 146 | 0.26 |
| 7A | 0.84 | 7.2 | 0.1500 | 0.12 | 922 | 0.52 | 151 | 0.57 |
| 7B | 0.85 | 10.2 | 0.2402 | 0.12 | 1200 | 0.29 | 140 | 0.24 |
| 8A | 0.84 | 6.1 | 0.1219 | 0.11 | 733 | 0.73 | 162 | 0.70 |

TABLE 5
Physical data

| | | BET | CTAB | d50 Median | | |
|---|---|---|---|---|---|---|
| | Oil | Surface | Surface | Particle | Pack | |
| Exam- | Absorption | Area | Area | Size | Density | 5% |
| ple | (cc/100 g) | (m²/g) | (m²/g) | (μm) | (g/mL) | pH |
| 6A | 87 | 83 | 59 | 7.9 | 0.61 | 9.2 |
| 6B | 90 | 50 | 48 | 6.6 | 0.54 | 10.1 |
| 7A | 103 | 87 | 80 | 17.7 | 0.45 | 10.0 |
| 7B | 96 | 76 | 69 | 7.0 | 0.50 | 10.2 |
| 8A | 97 | 46 | 46 | 9.2 | 0.45 | 9.7 |

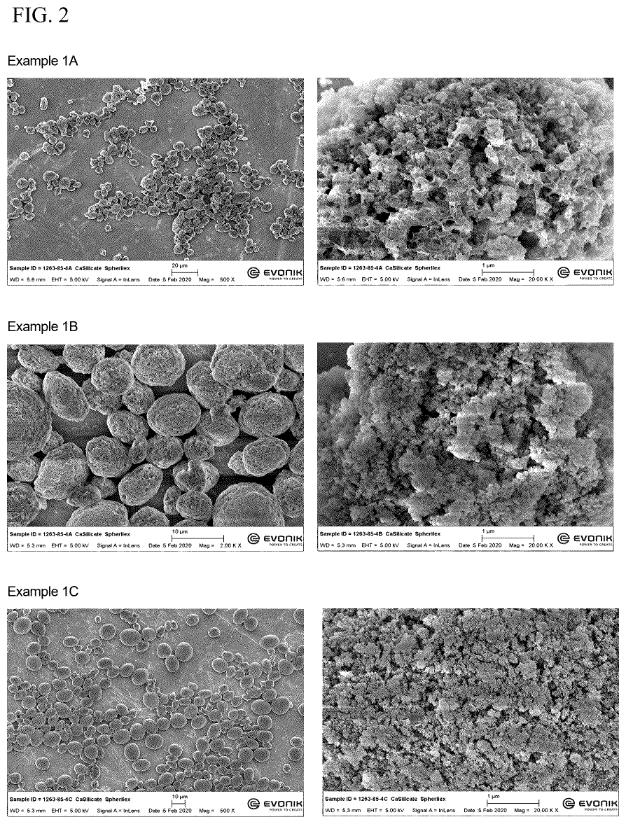

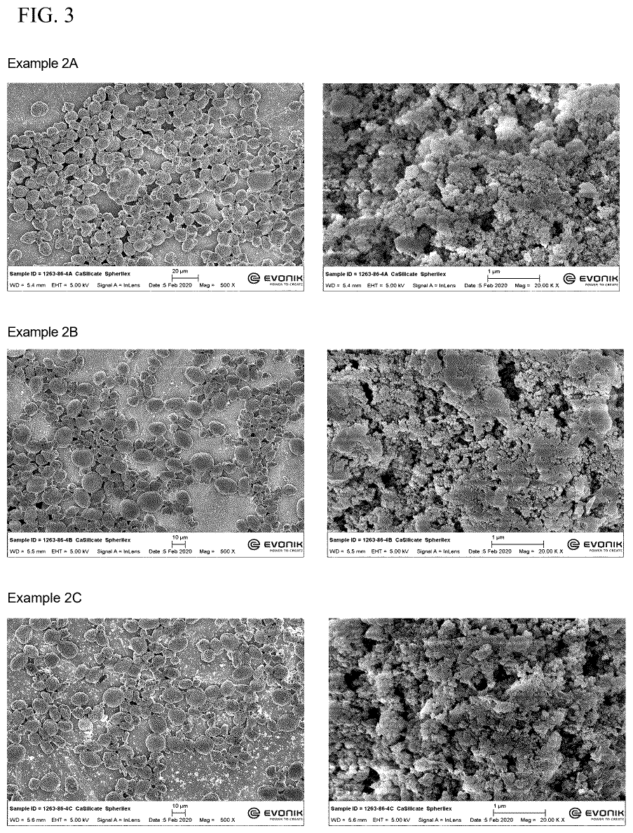

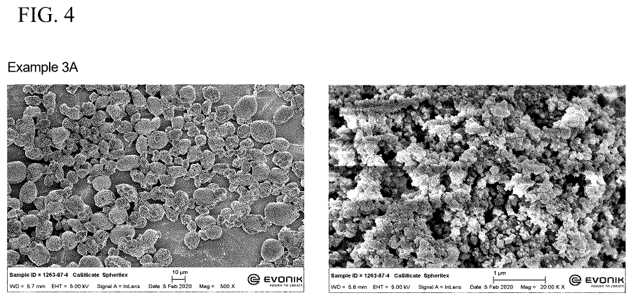

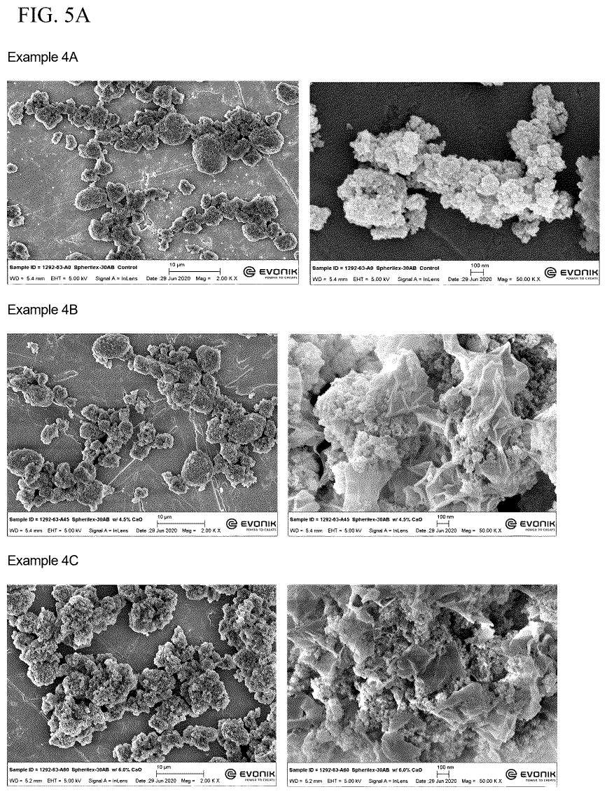

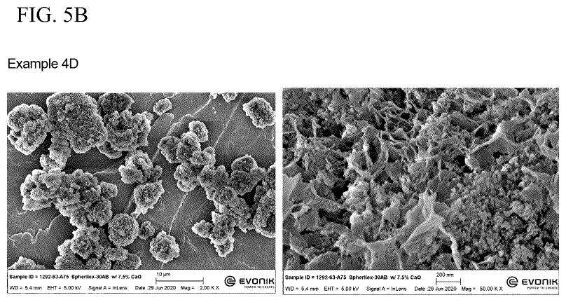

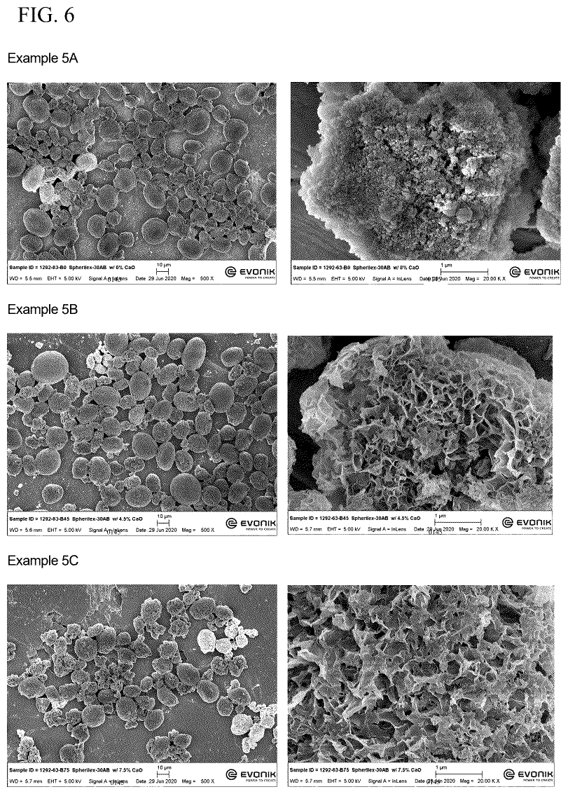

SEM images in FIGS. 2-6 show the spherical particle morphology of the calcium silicates. When looking at the surface of the inventive calcium silicate materials, a petaloid structure is sometimes observed. This petaloid structure is described in U.S. Pat. No. 6,287,530 section 5 line 56 as a structure like a flower of a rose that can be observed on the surface by electron microscopy. Not to be bound by a particular theory, but it is thought the petaloid structure may lead to higher levels of accessible calcium which may be beneficial for applications in oral care, cosmetics, coatings and food areas.

### Example 9: Toothpaste Formulation

In oral care, calcium can be used for remineralization, by increasing the soluble calcium levels in saliva and promoting the formation of new hydroxyapatite on the dentin or enamel surface. It can also possibly help small silica particles designed for tubule occlusion to have a higher affinity to the dentin surface and be more likely to remain in the mouth after brushing. Since these silicas are lower in surface area than traditional calcium silicate products, they can also provide mechanical cleaning to the tooth surface as well. PCR and RDA testing was performed at Indiana University School of Dentistry.

The toothpaste formulation and data is shown in the Table 6 below.

### Relative Dentin Abrasion (RDA)

The RDA values of dentifrice compositions of Examples DC1-DC14 containing the silicas of the current invention was determined according to the method set forth by Hefferen, Journal of Dental Res., July-August 1976, 55 (4), pp. 563-573, and described in Wason U.S. Pat. Nos. 4,340,583, 4,420,312 and 4,421,527, the contents of which are incorporated herein by reference in their entirety.

### Pellicle Cleaning Ratio (“PCR”)

The cleaning property of dentifrice compositions was typically expressed in terms of Pellicle Cleaning Ratio (“PCR”) value. The PCR test measures the ability of a dentifrice composition to remove pellicle film from a tooth under fixed brushing conditions. The PCR test is described in “In Vitro Removal of Stain with Dentifrice” G. K. Stookey, et al., J. Dental Res., 61, 12-36-9, 1982. Both PCR and RDA results vary depending upon the nature and concentration of the components of the dentifrice composition. PCR and RDA values are unit-less.

TABLE 6
Toothpaste Formulation (%)

| | | | | |
|---|---|---|---|---|
| Glycerin, 99.7% | 11.00 | 11.00 | 11.00 | 11.00 |
| Sorbitol, 70.0% | 40.00 | 40.00 | 40.00 | 40.00 |
| Deionized water | q.s. | q.s. | q.s. | q.s. |
| PEG-12 | 3.00 | 3.00 | 3.00 | 3.00 |
| Cekol 2000 | 1.20 | 1.20 | 1.20 | 1.20 |
| Tetrasodium | 0.50 | 0.50 | 0.50 | 0.50 |
| pyrophosphate | | | | |
| Sodium saccharin | 0.20 | 0.20 | 0.20 | 0.20 |
| Sodium fluoride | 0.24 | 0.24 | 0.24 | 0.24 |
| ZEODENT ® 165 | 1.50 | 1.50 | 1.50 | 1.50 |
| Example 6A | 10.00 | — | — | — |
| Example 6B | — | 10.00 | — | — |
| Example 7A | — | — | 10.00 | — |
| Example 7B | — | — | — | 10.00 |
| Titanium dioxide | 0.50 | 0.50 | 0.50 | 0.50 |
| Sodium lauryl sulfate | 1.20 | 1.20 | 1.20 | 1.20 |
| Flavor | 0.65 | 0.65 | 0.65 | 0.65 |
| Total | 100 | 100 | 100 | 100 |
| PCR | 83 | 78 | 78 | 79 |
| RDA | 167 | 153 | 147 | 132 |

### Example 10: Food Mixtures

Calcium silicates are also used as anti-caking and free flow aids in food applications. They are typically added to spice and/or food mixtures at levels in the 1-2% range to improve flow and handling properties during food preparation. A study was performed to show the efficacy of these materials as anti-caking and free flow aids in a table salt. Table salt was treated with 1.5% (double check) of each prototype calcium silicate and was thoroughly mixed. The salt samples were then exposed to moisture (chamber or added water) and were then tested for flowability. It was found that the salt control containing no calcium silicate had flow characteristics significantly worse than the salt samples containing the inventive calcium silicates. The data is show in the Table 7 below.

Angle of repose and compressibility testing were performed on a Hosokawa Micron Corporation, Summit, NJ, Powder Tester model PT-S according to the instructions provided with the instrument. Moisturized salt and moisturized salt containing 2% of the inventive calcium silicates by weight were evaluated.

Angle of repose testing was performed and lower slopes for the salt containing the calcium silicates was observed. The lower angle of repose values for the samples resulted from a decrease in particle to particle cohesion which is indicative of improved flowability.

Compressibility values were determined by the ratio of the aerated bulk density and the packed bulk density. Lower compressibility values result from salt samples containing calcium silicates which decreased the interparticle cohesiveness and allowed the salt to more efficiently fill given volume. It was found that all samples containing the inventive calcium silicates resulted in lower compressibility values and improved flowability.

TABLE 7

| Example | Angle of Repose (°) | Compressibility (%) |
|---|---|---|
| Control Salt | 48 | 17.3 |
| 6A | 43 | 11.8 |
| 6B | 44 | 12.8 |
| 7A | 45 | 11.9 |
| 8A | 42 | 12.3 |

### Example 11: Personal Care Application

Calcium silicates are also commonly used in personal care applications (U.S. Pat. No. 7,163,669). Since these calcium silicates have a spherical shape, they can be used for skin feel while also providing the ability to neutralize acidic odorants. Previous work has indicated that the ability to absorb acidic odors from sebum is an important functionality in deodorants and anti-perspirants. Samples were evaluated for their acid neutralization capacity by titration with HCl. Test calcium silicates were slurried at 5% by weight and mixed at 300 RPM in a ZetaProbe acoustic zeta potential instrument from Colloidal Dynamics. The instrument was equipped with and automatic pipette system that allows for accurate addition of liquid solutions. 1.0M HCl was added at 0.25 ml/min until the pH was stabilized at 4.0. The volume of acid required to reduce the pH to 4.0 is directly related to the neutralization capacity of the calcium silicate prototypes. The acid neutralization capacity is shown in Table 8 below.

TABLE 8

| Example | Volume of 1.0M HCl added (ml) |
|---|---|
| 6A | 9.67 |
| 6B | 36.4 |
| 7A | 35.6 |
| 7B | 52.4 |
| 8A | 28.6 |

### Example 12: Preparation of Liquid Coatings

### Solvent Borne Formulation Preparation (Examples 12A-12B)

Formulation Examples 12A and 12B were prepared by mixing the ingredients together for each component following the order listed in Table 9 at ambient room temperature. Then 2.5 mm ceramic grind media were added to the containers (1:1 by mass of media to total formulation). Formulations were ground on a LAU disperser for 2 h to achieve a Hegman score of 6 (25.4 micron). Grind media was separated using a 226 micron mesh filter.

TABLE 9
Solvent-borne High Silicone Formulation*

| | | Example | Example |
|---|---|---|---|
| Add Order | Component | 12A | 12B |
| Silikophen P80X | Silicone resin | 43.7 | 43.7 |
| Xylene | Solvent | 23.8 | 23.8 |
| Butyl glycol acetate | Solvent | 1.7 | 1.7 |
| Isobutanol | Solvent | 0.3 | 0.3 |
| TEGO Dispers 670 | Dispersant | 0.8 | 0.8 |
| TEGO Airex 900 | Defoamer | 0.4 | 0.4 |
| Aerosil 200 | Silica thixotrope | 0.6 | 0.6 |
| Bentone 38 | Clay thickener | 0.3 | 0.3 |
| Heucophos ZPO | ACP (Zinc phosphate) | 3.9 | — |
| Example 6B | ACP (Calcium | — | 3.9 |
| | silicate) | | |
| Ti Pure R 706 | TiO2 | 13.2 | 13.2 |
| Mica Suzorite 325 S | Mica | 11.6 | 11.6 |
| Total | | 100 | 100 |
| Percent Solids (% w/w) | | 65 | 65 |

*Formulations prepared using 3.9% corrosion inhibitor
ACP: anti-corrosion pigment

### Waterborne Formulation Preparation (Examples 12C-120)

Formulation Examples 12C and 120 were prepared similarly by first mixing the ingredients together for each component in A-pack (except for epoxy resin and thickener), as the order listed in Table 10 at ambient room temperature. It was ground with a cowl blade on a Dispermat for at least 30 minutes to achieve a Hegman score of 6 (25.4 micron). Then epoxy resin and thickener were added and mixed for another 10 minutes. Formulation B-pack was prepared by mixing curing agent and solvents before the coating's application.

TABLE 10
Water-borne 2k Epoxy Primer Formulation**

| | | Exam- | Exam- |
|---|---|---|---|
| Add Order | Component | ple 12C | ple 12D |
| A pack | | | |
| DI Water | Solvent | 10.9 | 10.9 |
| TEGO Dispers 757 W | Dispersant | 1.2 | 1.2 |
| Surfynol DF 75 | Defoamer | 0.4 | 0.4 |
| Surfynol 420 | Wetting Agent | 0.4 | 0.4 |
| Red Iron Oxide | Pigment | 6.4 | 6.4 |
| Zeeospheres G 400 | Ceramic Microspheres | 5.5 | 5.5 |
| Sparwite Barytes | Barium Sulfate | 5.5 | 5.5 |
| Wollastocoat 10ES | Calcium Inosilicate | 5.5 | 5.5 |
| Halox SW 111 | ACP (Zinc Free Corrosion | 8.5 | — |
| | Inhibitor) | | |
| Example 6B | ACP (Calcium silicate) | — | 8.5 |
| Mica 325 | Mica | 0.8 | 0.8 |
| Ancarez AR555 | Epoxy resin | 36.5 | 36.5 |
| Rheolate 310 (15% | Thickener | 2.8 | 2.8 |
| Solution in Water) | | | |
| B pack | | | |
| Anquamine 419 | Curing Agent | 10.0 | 10.0 |
| Propylene Glycol | Solvent | 2.9 | 2.9 |
| methyl ether | | | |
| DI Water | Solvent | 2.7 | 2.7 |
| Total | | 100 | 100 |
| Percent Solids | | 60 | 60 |
| (% w/w) | | | |

*Formulations prepared using 8.5% corrosion inhibitor
ACP: anti-corrosion pigment

### Coating Application

Solvent borne coatings were applied at 3 mil wet film thickness using drawdown bar to 4×6 cold rolled steel panels and allowed to flash overnight in ventilation. Samples were baked at 250 C for 30 minutes.

Waterborne coatings were applied at 6 mil wet film thickness using drawdown bar to 4×6 cold rolled steel panels and allowed to cure at ambient condition for 7 days.

### Testing of Cured Coating

Both solvent borne and waterborne coatings were evaluated in a similar way. Panels were scribed using box cutter blade according to ASTM D1654. Exposed metal edges and back of panels were covered with vinyl tape.

Solvent borne samples were submitted for 300 h salt fog corrosion testing, while waterborne samples were submitted for 1000 h salt fog corrosion testing. After reaching 300 h (for solvent borne) and 1000 h (for waterborne samples), panels were removed and the surfaces were wiped dry. Panels were scraped using metal spatula and eight scribe creep measurements were taken uniformly along the scribe according to ASTM D1654. The average scribe creep was the average of the eight measurements.

Table 11 shows the result of the scribe creep measurements.

TABLE 11

| | Comparison | Inventive | Comparison | Inventive |
|---|---|---|---|---|
| | Exam- | Exam- | Exam- | Exam- |
| | ple 12A | ple 12B | ple 12C | ple 12D |
| Average Scribe | 1.15 | 1.20 | 1.70 | 1.65 |
| Creep | | | | |
| [mm from scribe | | | | |
| center] | | | | |

The inventive examples (12B and 12D) show comparable anti-corrosion performance against current commercial benchmark anti-corrosion pigments (12A and 12C).

Additional the inventive examples have an environmental benefit for the calcium silicate compared to the use of the traditional additives phosphates.

1. A spherical calcium silicate having
(i) a d50 median particle size in a range from 1 to 35 μm,
(ii) an oil absorption in a range from 40 to 130 mL/100 g,
(iii) a S₈₀ sphericity factor of greater than or equal to about 0.80,
(iv) a BET surface area of between 10-125 m²/g,
wherein the spherical calcium silicate comprises
(v) an amount of CaO from 0.5-20 wt-%.

2. The spherical calcium silicate according to claim 1, wherein a cetyltrimethylammonium bromide surface area is between 5 and 80 m²/g.

3. The spherical calcium silicate according to claim 1, wherein a pack density is between 0.32 and 0.96 g/mL.

4. The spherical calcium silicate according to claim 1, wherein a 5% pH is between 8.0 and 12.0.

5: The spherical calcium silicate according to claim 1, wherein a water absorption is in a range from 55 to 115 mL/100 g.

6. The spherical calcium silicate according to claim 1, wherein a Ca:Si weight ratio is between 0.015 to 0.600.

7. A process for the production of spherical calcium silicate according to claim 1, the process comprising:
(a) continuously feeding a mineral acid and an alkali metal silicate into a loop reaction zone comprising a stream of liquid medium, wherein at least a portion of the mineral acid and the alkali metal silicate react to form a silica product in the liquid medium of the loop reaction zone;
(b) continuously recirculating the liquid medium through the loop reaction zone;
(c) continuously discharging from the loop reaction zone a portion of the liquid medium comprising the silica product;
(d) filtrating and washing the liquid medium comprising the silica product;
(e) combining a filter cake of the filtrating in (d) with calcium hydroxide,
(f) stirring the combined filter cake and calcium hydroxide of (e) for 10 min to 180 min, and
(g) drying a solution comprising the combined filter cake and calcium hydroxide.

8. The process for the production of spherical calcium silicate according to claim 7, wherein feed locations of the mineral acid and the alkali metal silicate into the loop reaction zone are different.

9. The process for the production of spherical calcium silicate according to claim 7, wherein a total feed rate of acid and silicate in (a) is proportional or equal to a discharge rate of the liquid medium containing the silica product in (c).

10. The process for the production of spherical calcium silicate according to claim 7, wherein in (b) all or substantially all of the contents within the loop reaction zone are recirculated at a rate ranging from 50 vol. % per minute to 1000 vol. % per minute.

11. Dentifrice compositions, food mixtures, personal care applications and liquid coatings comprising the spherical calcium silicate according to claim 1.

12. A dentifrice composition comprising the spherical calcium silicate of claim 1.

13. A coating composition comprising the spherical calcium silicate of claim 1.

14. A food composition comprising the spherical calcium silicate of claim 1.

15. A cosmetic composition comprising the spherical calcium silicate of claim 1.
